(19) United States
(12) Patent Application Publication
(10) Pub. No.: US 2003/0195423 A1
(43) Pub. Date: Oct. 16, 2003

(54) Device for determining a suitable location for catheterized delivery of medications

(76) Inventors: David E. Gibbs, Ottawa (CA); Neil Teitelbaum, Ottawa (CA)

Correspondence Address: Neil Teitelbaum, 834 Colonel By Drive, Ottawa, ON, K1S 5C4, CA

(21) Appl. No.: 10/418,720

(22) Filed: Apr. 16, 2003

Related U.S. Application Data
(63) Continuation-in-part of application No. 09/494,632, filed on Jan. 31, 2000.

(51) Int. Cl. A61B 8/00

(52) U.S. Cl. 600/438; 604/22

(57) ABSTRACT

An apparatus and method for determining a suitable location for catheterized delivery or infusion of medication are disclosed. The apparatus includes a probe having a rod coupled to a transducer. The transducer introduces an acoustic or incident wave into the rod. The wave travels through the rod to a tissue in a patient's mouth about the end of the rod, and at least part of the wave is reflected by the tissue back into the rod towards the transducer. The reflected wave contains information relating to the structure of the tissue beneath the probe's tip. The differences between successive oral readings are used to distinguish between different regions of tissue (e.g. containing bone tissue, tooth, ligament, etc.) and/or is used to analyze the density of a particular region, for finding a suitable location for inserting a drill/catheter.

### RELATED APPLICATIONS

This application is a continuation in part of patent application Ser. No. 09/494,632 filed Jan. 31, 2000.

### FIELD OF THE INVENTION

The present invention provides for a simple method and device for determining a suitable location for infusing or injecting medication.

### BACKGROUND OF THE INVENTION

There are a variety of methods currently in use for providing local anaesthetic in dentistry. These methods and apparatuses however all have disadvantages, either being difficult for practitioners to perform or painful and unpleasant to the patient.

An example of a method used currently in dentistry is the infiltration method, whereby a local anaesthetic solution is injected into the soft tissue of gingiva. In doing so, the solution eventually passes through the cortical plate affecting the nerve bundle entering the tooth. Disadvantages of this method include the delay of onset of anaesthesia after the injection and, in most cases, ballooning of the injected tissue. As well, there is an extended period of time for recovery of the tissue until return to normal condition.

Another method that is currently used is the regional block method whereby an anaesthetic solution is injected locally in proximity to the nerve trunk as it enters the bone. Disadvantages of this procedure are that it is extremely difficult to locate the nerve trunk, there is discomfort to the patient and a delay for the anaesthetic to take effect. As in the case of the infiltration method, this method necessitates a long recovery period for tissue to return to normal.

At present, two types of apparatus have been used to perform intra-osseous anaesthesia. These are surgical burs used to perforate the cortical plate and the villet injectors.

The use of a surgical bur has disadvantages in that burs are expensive and they have to be sterilized between uses or a new bur used each time. In addition, the method is slow, requiring the attached gingiva and periosteum to be anaesthetized before the cortical plate is perforated. The villet injector is an apparatus that serves both as a perforator and injector. It uses specially designed needles rotated by a conventional dental motor. A disadvantage of this device is that the needle often becomes clogged with pulverized bone, which obstructs the passage in the needle and prevents injection of the anaesthetic solution. It is generally difficult to remove the clogging material from the needle and often the use of a second needle is necessary. Other disadvantages of this method include the initial capital cost of the instrument purchase, and the cost of the needles that are somewhat expensive. In addition, the design of the instrument makes access to various parts of the mouth difficult and sometimes impossible.

Intra-osseous and targeted root-canal nerve anaesthesia has not become popular for the reason that there has not been a practical technique of making the injections successfully. For example, there has been a general belief that this method is radical and to be resorted to only if nerve block and infiltration anaesthetic do not accomplish the desired result. However, intra-osseous and targeted injections produce positive, more profound anaesthesia and could be made with less pain than either of the other types according to the present invention.

Targeted anaesthesia has several advantages over prior art nerve block or infiltration methods. There is no feeling of numbness in the tongue, cheek, or lips during or after the injection and there is no after-pain. The anaesthetic is profound and acts immediately alleviating the necessity of waiting for the anaesthetic to take effect as with the nerve block and infiltration methods.

Furthermore, as only a few drops of anaesthetic are injected, there is no feeling of faintness or increasing of the pulse rate.

To achieve targeted anaesthesia one must gain access, if intra-osseous, to the cancellous bone by going through the cortical layer; or to the bottom of the tooth, if root-canal targeted anaesthesia is desired. Because of instant anaesthesia and profound pulpal anaesthesia, there is much greater control over the region one wishes to anaesthetize, resulting in a much smaller dose of anaesthetic; as well as, of course, other medication, where applicable.

U.S. Pat. No. 5,779,708 in the name of Wu filed Aug. 15, 1996 and issued Jul. 14, 1998, and U.S. Pat. No. 5,762,639 in the name of Gibbs filed Jun. 6, 1995 and issued Jun. 9, 1998, both incorporated herein by reference, disclose an apparatus and method for catheterized delivery or infusion of medication and anaesthesia. In both patents a perforating catheter is first used to perforate the periodontal ligament and/or the cortical plate of bone tissue, and is then left in place and used as a catheter for insertion of a hypodermic needle of smaller gauge to deliver medication or anaesthesia to a target area. The perforator is a bevelled needle for drilling into the ligament or bone tissue. For drilling, the device comprises an adaptor that transmits the rotational movement from a dental hand piece or the like to the bevelled needle. The adaptor may have a rod that extends axially into the bevelled needle when the device is assembled for drilling. The rod is used to prevent the debris resulting from drilling from blocking the passage in the bevelled needle. As well, the rod reinforces the needle and maintains the alignment between the perforator and the adaptor for improved drilling efficiency.

Over the past 50 year or so, devices and processes for intra-osseous anesthesia have been developed and refined. However, prior to the invention of the applicant Gibbs, no other inventors have provided a useful, workable convenient and inexpensive solution that affords all of the benefits provided by Gibbs' invention. For example, non of the prior art devices allow a motorized hand-piece to drive a small intra-osseous catheter/drill having a rod/drill therein, wherein the device is placed by drilling at high or slower speeds and removed by simple withdrawal by pulling out the catheter. Most of the effort in this field had been directed toward longer term delivery of medication wherein the catheters have had some means of latching into the bone for more permanent placement. Furthermore, the invention described in the aforementioned Gibbs patent does not suffer from many of the drawbacks of inserting the needle/drill into the bore being cut by the end tip of the drill, since the outside walls of the needle/drill are of a uniform diameter and non-varying.

Although U.S. Pat. No. 5,779,708 and U.S. Pat. No. 5,762,639 describe a device that is believed will revolutionize dentistry, or at least local anesthesia in the field of dentistry, there remains an enhancement to the system which this invention addresses.

The drill/catheter described by both Gibbs and Wu works effectively if used correctly, however, the possibility exists that the drill/catheter may be placed in a poor or incorrect location for insertion. For example, it is desirable that the drill be placed in the soft bone adjacent to the tooth or teeth requiring anesthetic. If the drill/catheter is accidentally directed at the tooth itself, it will likely break and successful delivery of local anesthesia will not result. Unnecessary breakage of the drill/catheter makes the procedure unduly expensive and time consuming.

Furthermore, the possibility of having to repeat the drilling procedure due to incorrect placement of the drill/catheter, makes the patient uncomfortable and less confident with the dental procedure and/or practitioner. The latter is of particular importance, since wisdom and expertise varies amoung practitioners.

It would be advantageous to provide an apparatus and method that allows the practitioner to quickly and accurately determine a suitable location for inserting the drill/catheter.

It is an object of this invention to provide a probe that will indicate a suitable location for insertion of the drill/catheter into the soft bone adjacent the tooth itself.

It is an object of this invention to provide a portable device for achieving this end.

It is the belief of the inventor, that this novel method and combination of elements will eventually change the way in which many dentists infuse medication and local anesthesia.

### SUMMARY OF THE INVENTION

The present invention endeavours to mitigate the problems and disadvantages of delivering dental anaesthetic encountered with the prior art methods and devices.

In accordance with the invention, there is provided, a probe for providing indicia related to the density of bone under investigation within a patient's mouth about a region of a tip of the probe, and for determining a suitable location for delivery of anesthesia.

In accordance with the invention, there is provided, a probe for determining a suitable location for positioning a drill/catheter for delivery of medication comprising: an end for placing within a patients mouth adapted to be coupled with a tissue within the patients mouth; a transducer for providing an acoustic wave to the tissue and for receiving acoustic waves reflected from the tissue; and, an electronics module for determining relative density measurements related to the reflected acoustic waves, the relative density measurements providing at least indicia related to a difference of two different locations representing two different tissue densities in two different scanning regions.

In accordance with the invention, there is provided, a probe for providing an indication of the density of bone under investigation within a patient's mouth about a region of a tip of the probe, and for determining a suitable location for delivery of medication, said probe comprising: an acoustic coupler having an end corresponding to the tip of the probe for placing in contact with a surface about the bone; a transducer coupled to the acoustic coupler for converting electrical signals into mechanical vibrations for being transmitted to the surface, and for converting reflected mechanical vibrations into electrical signals indicative thereof; and, means for providing at least one relative measurement related to the reflected mechanical vibrations, the reflected mechanical vibrations containing information related to the density of bone under investigation within the patient's mouth, each relative measurement determined from a difference between a first reading and a second reading, the first and second readings obtained from first and second regions, respectively, at least one relative measurement for providing indicia related to the suitable location for delivery of medication.

In accordance with the invention, there is provided, a probe for placement in a patient's mouth for determining a suitable location for inserting a catheter/drill for delivery of medication, said probe comprising: a rod having a driving end and a sensing end; a transducer coupled to the driving end of the rod, for imparting mechanical energy into the driving end of the rod, which is transmitted though the rod to a tissue about the sensing end of the rod and is reflected by the tissue as a reflected wave traveling through the rod towards the driving end, for analyzing the reflected wave, and for emitting electrical signals indicative thereof; and, a controller coupled to the transducer, for processing the electrical signals and for determining a difference between two successive measurements for obtaining a relative measurement indicative of the structure of the tissue about the sensing end.

In accordance with another aspect of the invention, a method is provided of obtaining relative density measurements relating to the density of tissue in an individual's mouth using a hand-held acoustic probe for determining a suitable location for performing a dental procedure, said method comprising the steps of: placing the probe about a first region in the individual's mouth for acquiring a first reading; placing the probe about a second region in the individual's mouth for acquiring a second reading, the second region in close proximity to the first region; determining a difference between the first and the second readings, the difference indicative of a difference in density between the first region and the second region; and, determining the suitable location in dependence upon the determined difference between the first and the second readings.

In accordance with the invention, there is further provided, a method of determining a suitable location for positioning a drill/catheter for delivery of medication comprising the steps of: providing a probe capable of providing relative density measurements, the relative density measurements providing at least indicia related to a difference of two different locations representing two different tissue densities in two different scanning regions; placing the probe at at least two scanning regions and allowing the probe to scan the at least two regions; after receiving an indication of a suitable location for drilling, inserting the drill/catheter about the suitable location.

In accordance with the invention, there is further provided, a method for determining a suitable location for delivery of anesthesia to a predetermined region within a patient's mouth, comprising the steps of: placing a probe having a rod with a driving end and a sensing end in the patient's mouth, the sensing end in contact with a tissue about the predetermined region; imparting an incident wave into the driving end so that the incident wave travels longitudinally along the rod to the tissue in contact with the sensing end where at least a portion of the incident wave is reflected to form a reflected wave travelling back towards the driving end; measuring acoustic characteristics of the reflected wave and emitting electrical signals indicative thereof; processing the electrical signals and comparing the electrical signals to other measured electrical signals for establishing a relative measurement indicative of a change in tissue structure; and, determining the suitable location for delivery of anaesthesia in dependence upon the relative measurement.

In accordance with the invention a method of determining a suitable target location for inserting an intaosseous device is provided comprising the steps of:

placing the probe about a first region in the individual's mouth for acquiring a first reading;

placing the probe about a second region in the individual's mouth for acquiring a second reading, the second region in close proximity to the first region;

determining a difference between the first and the second readings, the difference indicative of a difference in density between the first region and the second region; and

determining the suitable location in dependence upon the determined difference between the first and the second readings.

In accordance with yet another embodiment a method of determining a suitable location for inserting an intraosseous device is provided comprising the step of:

placing an electronic a probe about a plurality of locations along an upper or lower gum within a small region substantially between two teeth to determine a location where the density is the least, said location being a target location where it is likely to find the absence of a tooth under the gum.

### BRIEF DESCRIPTION OF THE DRAWINGS

FIG. 1 illustrates an exploded view of a prior art drill/catheter showing the component parts and their inter-relationship;

FIGS. 2A-2C illustrate a prior art method using the device of FIG. 1; FIG. 2A shows the device drilling in the bone tissue; FIG. 2B shows the perforator inserted into the bone tissue and the adaptor de-coupled; and FIG. 2C shows the perforator inserted into the bone tissue as a catheter and a hypodermic needle set for delivering an injection;

FIG. 3 illustrates an alternative prior art method of delivery medication to treat a root-canal nerve;

FIG. 4 illustrates from a plan view the point of catheter insertion for the alternative method shown in FIG. 3;

FIG. 5 is a cross-section a view of a probe for determining a suitable location for injecting medication via a drill/catheter according to one embodiment of the invention;

FIG. 6 is a block diagram of the electronics module coupled to the probe of FIG. 5;

FIG. 7 is a flow chart of the digital signal processor and A/D functions;

FIG. 8 is a perspective view of the probe for determining a suitable location for injecting medication according to another embodiment of the invention;

FIG. 9 is a cross-sectional/perspective view of the probe for determining a suitable location for injecting medication according to another embodiment of the invention;

FIG. 10 is a cross-sectional view of the probe for determining a suitable location for injecting medication according to another embodiment of the invention; and

FIG. 11 is an exploded/perspective view of the probe for determining a suitable location for injecting medication according to another embodiment of the invention.

### DETAILED DESCRIPTION OF THE PREFERRED EMBODIMENT

FIG. 1 illustrates an exploded view of a prior art drill/catheter device. The device comprises a perforator 1, an adaptor 3, and a cap 5.

The perforator 1 has a bevelled drilling needle 7 that is used both as a drill and a catheter. Needle 7 is bevelled at both ends. The first end 9 is formed as a drilling tip in that it has cutting teeth along the edge. The second end 11 is bevelled for receiving and directing the needle of a hypodermic syringe and for easy coupling with the adaptor 3. When rotated, the drilling needle 7 penetrates in the bone tissue through gingiva or ligament and drills a hole with the cutting tip 9. The perforator 1 remains in place as a catheter, with the drilling needle inserted into the bone. A hypodermic needle is introduced through the passage of drilling needle 7 to inject a medicament directly into the bone. Therefore, the drilling needle 7 is selected to have a wide enough passage for allowing a hypodermic needle with a smaller gauge to be inserted through needle 7.

The adaptor 3 conveys the rotational movement from a dental hand piece or the like to the perforator. As well, the adaptor comprises rod 27, which when inserted within the needle passage, advances through the length of the needle up to the bevelled end. In this way, the debris from drilling cannot penetrate to block the needle passage. In addition, the rod gives additional rigidity, strength, and alignment to needle 7 during drilling.

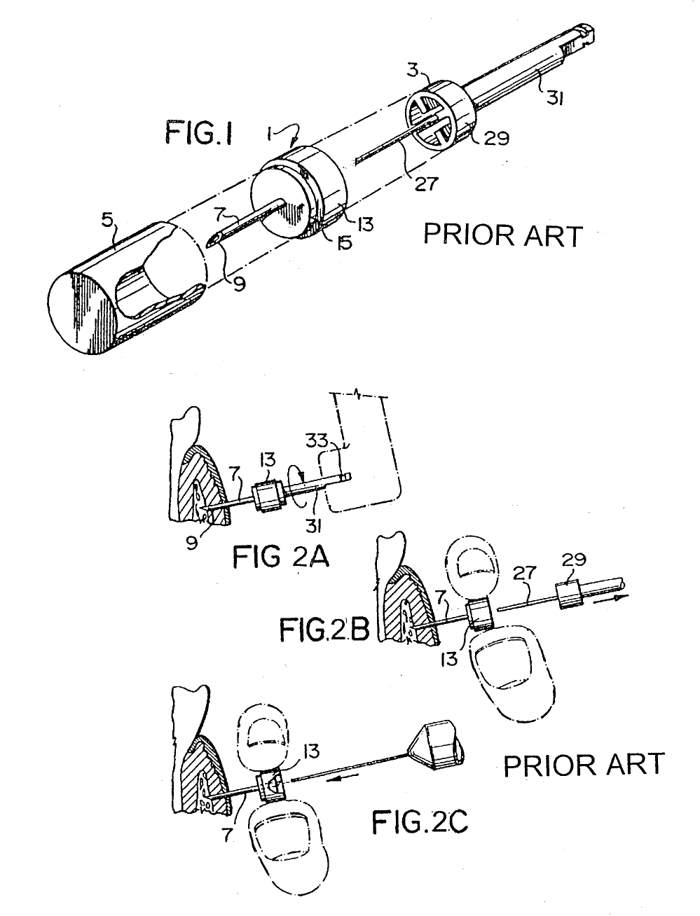

Referring to prior art FIGS. 2a-2c, the operation of the prior art device is as follows:

First, a site for the injection is selected by the practitioner. The gingiva over the injection site is disinfected and topically anaesthetized. A small amount of anaesthetic solution is injected until blanching of the tissue. This will anaesthetize the gingiva and the periosteum.

As can be seen in FIG. 2A, the bevelled end 9 of the needle 7 is placed against the gingiva and shank 31 is attached with joint 33 to a contra angle or to a straight dental hand piece. The adaptor and perforator are coupled for drilling.

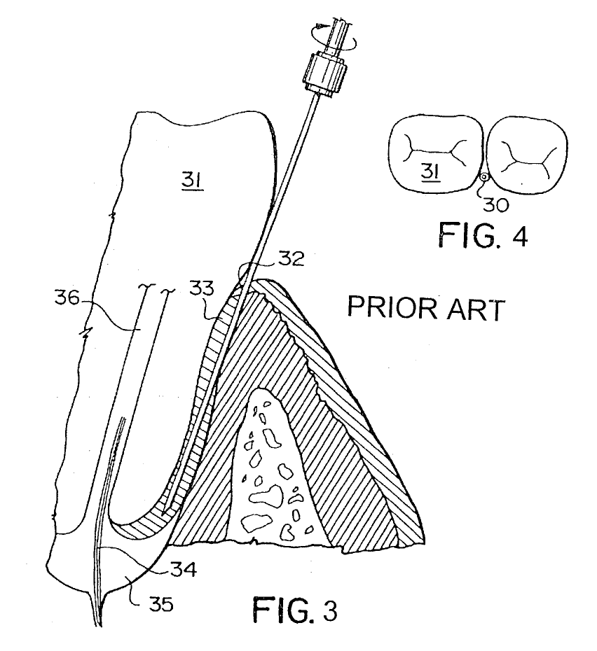

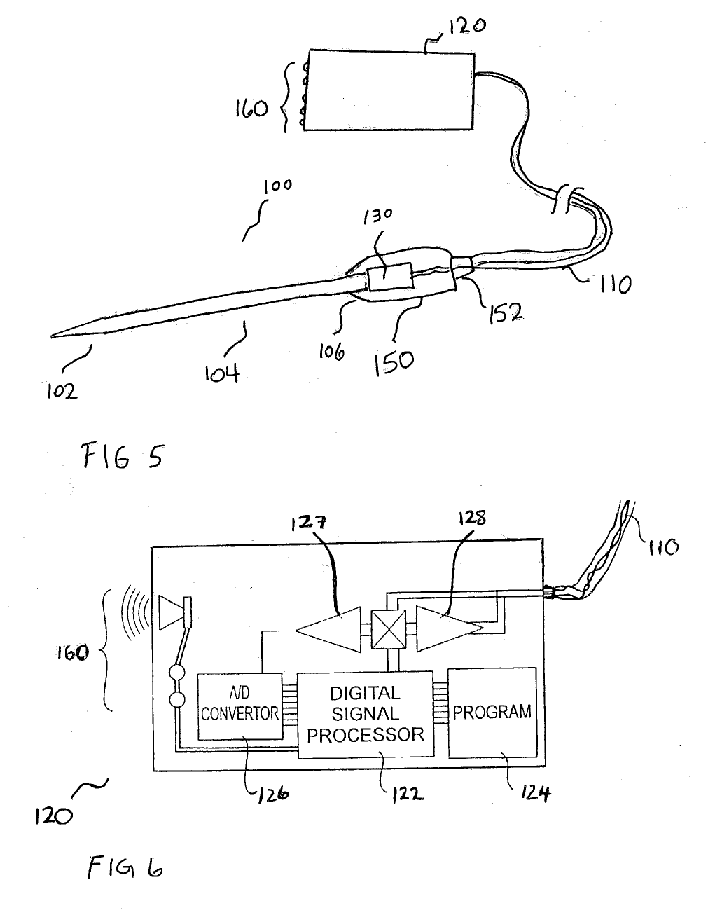

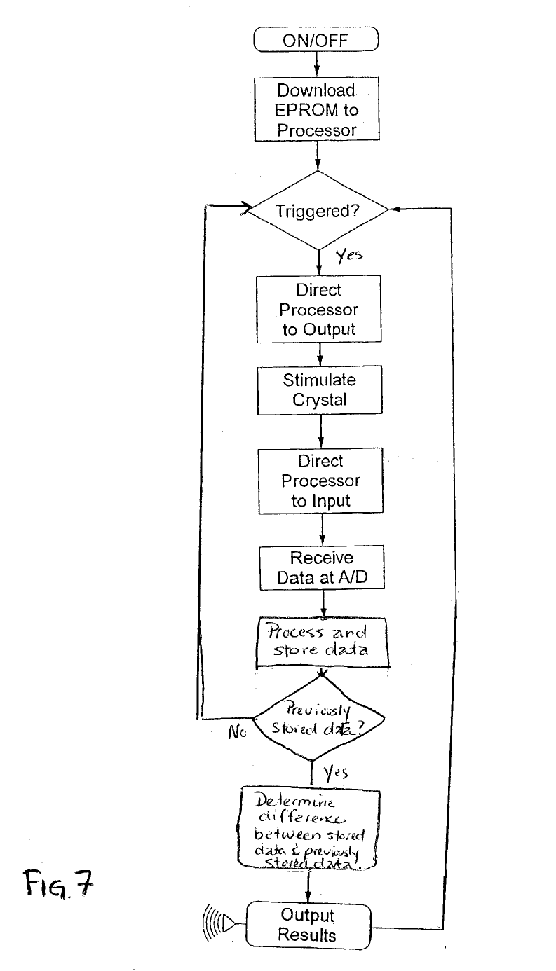

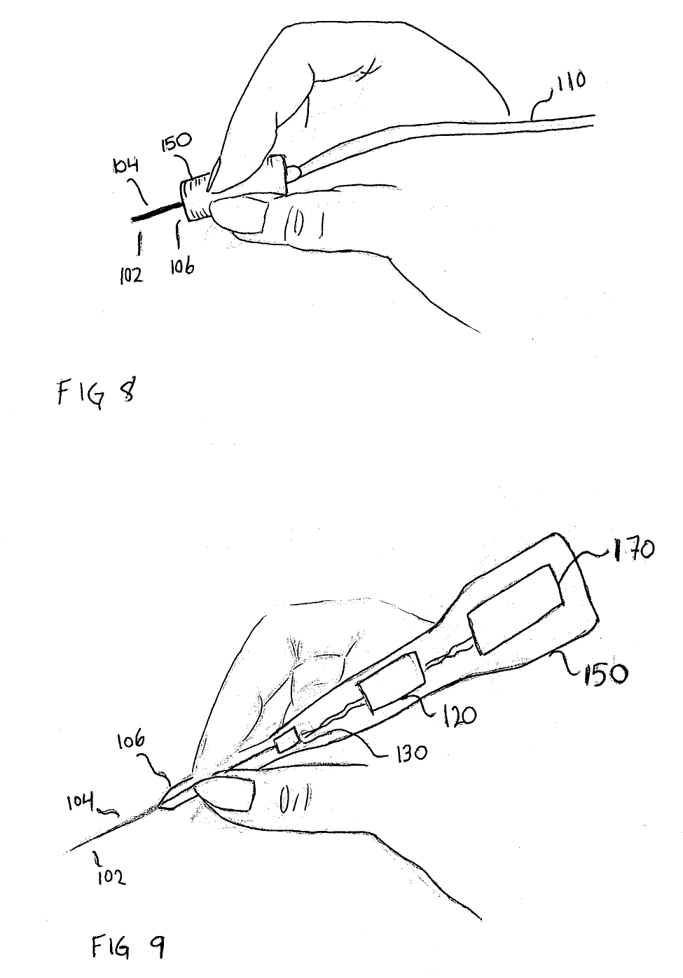

The perforator should be held perpendicular to the cortical plate, or if not possible or convenient, it should be held vertical and parallel to the long axis of the tooth as shown in FIG. 3, having been inserted between teeth as shown in FIG. 4. The perforator is then operated in small bursts of rotation from the hand piece until resistance is no longer felt, as is well known to dentists.

Next, the adaptor 3 is removed from the engagement with perforator 1 by applying pressure to the body 13 with the fingers thus keeping the needle 7 in the perforated cortical plate. This is shown in FIG. 2B.

The presence of the needle 7 in the cortical plate, or down the side of the tooth as in FIG. 3, allows an injection to be made without complicated manoeuvres to find the perforation in the case of floating gingiva or the free or marginal gingiva. FIG. 2C illustrates the next step, namely how the injection needle is inserted through the perforator 1 for delivering the anaesthetic solution required.

The last step is to remove the perforator 1 from the cortical plate and reinstall the cover cap 5 over the needle 7, then insert the adaptor to the perforator making the unit complete and disposable.

Although the device described heretofore and shown in the figures performs satisfactorily, and disposing the drill/catheter into the bone at a desired location is relatively simple, it is would be advantageous to provide a more accurate and reliable method of determining the desired location. Specifically, it would be advantageous to provide a method and device to determine a suitable location for inserting the drill/catheter or similar device for intra-osseous or targeted anaesthesia. More specifically, it would be advantageous to provide a hand-held probe that uses the inherent physical differences between tissue samples (e.g. bone tissue, tooth, ligament, etc.) in a patient's mouth, to determine a suitable location for delivering anaesthesia.

U.S. Pat. No. 5,754,494 to Gallagher, hereby incorporated by reference, discloses a hand-held prodder capable of distinguishing inert rock from potentially hazardous landmines. The prodder comprises a rod that is placed in contact with an object. A high frequency acoustic or incident wave is introduced into the rod and travels along the rod to the object where it is reflected back towards the piezoelectric crystal. The piezoelectric crystal converts the reflected wave to an electric signal and a signal processor determines values representative of the frequency-time-amplitude characteristics of the object. By comparing reflected wave characteristics to pre-determined characteristics for known materials, inert rocks and potentially hazardous plastic or metallic objects are distinguishable.

The device of the present invention is a hand-held probe based on similar principles to the device of U.S. Pat. No. 5,754,494. Specifically, the device of the present invention uses a hand-held probe that introduces an acoustic incident wave into a rod of the probe that is placed in contact with a tissue in a patient's mouth. The reflected wave contains information relating to the structure of the tissue beneath the probe's tip. The differences between successive oral readings are used to distinguish between different regions of tissue (e.g. containing bone tissue, tooth, ligament, etc.) and/or are used to analyze the density and/or porosity of a particular region. The relative measurements are used to find a suitable location for inserting a drill/catheter.

FIG. 5 illustrates the hand-held probe according to an embodiment of the present invention. The probe 100 is designed for at least a portion of the probe 100 to be easy placed within the mouth of a patient. The probe 100 includes a rod 104 having a sensing end 102 on one side and a driving end 106 on the other side. Preferably, the sensing end 102 is tapered, or at least of small diameter, to sense an area in the patient's mouth that is relatively localized. The driving end 106 is coupled to a transducer, e.g., in the form of a piezoelectric crystal 130. More specifically, the rod 104 directly abuts, is glued to, or is connected via a ceramic insulator or other material to an end of the piezoelectric crystal 130. The rod 104 is constructed of an appropriate material, such as a stainless steel, for transmitting acoustic waves. Housing 150 protects the piezoelectric crystal 130 and supports the driving end of the rod 106. Housing 150 also provides a ‘handle’ for which a user is able to grasp the probe 100. Positive and negative electrical leads 110 from the piezoelectric crystal 130 exit the housing 150 through connector 152 for bi-directional electrical signal transmission between the piezoelectric crystal 130 and a controller or electronics module 120.

The electronics module 120 is capable of two modes: a driver mode and a signal-processing mode. In the driver mode, an electrical signal is transmitted along leads 110 to the piezoelectric crystal 130 for generating a piezoelectric mechanical pulse, which is introduced into the rod's driving end 106. In the signal-processing mode, any electrical signals generated by the piezoelectric crystal 130 are transmitted along leads 110 for processing by the electronics module 120.

Referring to FIG. 6, the electronics module 120 includes a processor 122, an EPROM 124 containing program instructions, an A/D converter 126, a signal input amplifier 127, and a driver output amplifier 128. The electronics module 120 also includes an audio and/or visual output device 160.

A method of using the device, according to an embodiment of the present invention, is outlined in FIG. 7. The user activates the probe 100 and inserts at least a portion of the probe 100 into the patient's mouth. The user directs the sensing end 102 of the probe towards a predetermined area selected about a tooth/teeth requiring anaesthesia. The user places the sensing end 102 in contact with the tissue about the predetermined area.

When the probe 100 comes in contact with the tissue, such as the gingiva near the predetermined area, the probe 100 is automatically triggered to send an acoustic incident wave to the tissue via the rod 104. Specifically, the EPROM 124 signals the driver output amplifier 128 to generate an ultrasonic analog driver signal, which stimulates the piezoelectric crystal 130 to generate a mechanical pulse that is sent as an acoustic incident wave down the longitudinal axis of the rod 104.

A portion of the incident elastic wave reflects from the tissue about the rod's sensing end 102 and returns to the rod's driver end 106 as a reflected wave. Another portion of the incident elastic wave penetrates a portion of the tissue before it is reflected back to the rod's sensing end 102, and returned to the rod's driver end 106. Specifically, the elastic wave is reflected back to the rod where there are interfaces between tissues having different acoustic impedances, e.g. bone and tooth.

The mechanical energy in the reflected wave stimulates the crystal 130 to generate electrical analog signals characteristic of the reflected wave. The analog signals characteristic of the reflected wave are processed through the signal input amplifier 127 and converted by the A/D converter 126 for analysis by the processor 122. For example, the processor is able to perform discrete Fourier transforms to convert the reflected data from the time domain to the frequency domain. The processor 122 stores the reflected data in its RAM memory.

The reflected data contains information pertaining to the structure of the tissue in the predetermined region. For example, the reflected data from a high-density region will be different from the reflected data from a low-density region. Moreover, the reflected data contains information pertaining to the location of interfaces between different tissues.

Specifically, after the incident acoustic wave is transmitted from the sensing end to the tissue contacting the sensing end, at least a portion of the wave further penetrates the tissue below the probe 100. Depending on the density of the tissue, the incident wave will travel at a specific speed. When the incident wave encounters an interface, at least a portion of the wave is reflected back towards the sensing end. The time it takes the wave to return to the sensing end 102 of the rod 104 is dependent on the density of the intermediate tissue between the sensing end 102 and the reflecting surface. Accordingly, the reflected wave contains information relating to the depth structure of the tissue. The use of ultrasound for determining the density of bone is well known. U.S. Pat. Nos. 5,840,029 and 5,564,423, hereby incorporated by reference, are two examples.

While the probe 100 maintains contact with the tissue, either at a specific location or as it is moved along different regions of tissue, it is regularly triggered to transmit acoustic incident elastic waves. Alternatively, the probe 100 is triggered when the practitioner depresses an actuator, for example, an actuator in the form of a switch, a button, or a foot petal.

A reading is obtained each time the probe 100 is triggered. Specifically, each time the probe 100 is triggered an acoustic incident wave is transmitted down the longitudinal axis of the rod 104, is reflected from the tissue about the rod's sensing end 102, is returned to the rod's driver end 106 as a reflected wave, and an analog signal characteristic of the reflected wave is generated and is digitally converted to the reflected data. Thus, a plurality of readings is acquired as the probe 100 is slowly slid across a sample area, such as the gingiva in a patient's mouth.

At least a portion of the reflected data from each reading, other than a first reading, is compared to an analogous portion of data from a previous reading, to determine a relative measurement. The at least a portion of the reflected data selected from at least one of a frequency, time and amplitude of the reflected wave. For example, the amplitude at a particular frequency is monitored and compared to determine the relative measurement. Alternatively, the entire waveform of the reflected data is compared to the entire waveform of a previous measurement, the difference between measurements indicative of a change in tissue. Further alternatively, a phase change is monitored and compared.

In this way, the tissue structure in a predetermined region is measured as a difference between two measured readings. Since it is the difference between readings that is important, there is no need to calibrate the device. The difference in the readings is expressed via the audio/visual output device 160, i.e., a device that provides a plurality of distinctive audible tones and/or a plurality of LEDs or similar indicators. Alternatively, a LCD, CRT, or a similar indicator is used to provide a visual interpretation of the data, e.g., in the form of a graph or other image.

For example, when determining a suitable location for inserting a drill/catheter for targeted anaesthesia it is desirable to place the drill/catheter in the soft bone adjacent the tooth or teeth requiring anesthetic. Accordingly, the probe 100 is used to avoid drilling into a tooth. In such cases, it is desirable for the probe 100 to act in the same manner as a ‘stud finder’, wherein an audio signal indicates sharp transitions between tooth and bone tissue. For example, the intensity of an alarm increases or decreases according to the difference in the measurement. Specifically, a large difference in successive readings is accompanied with a loud audible signal and a small difference in successive readings is accompanied with a quieter or absent audible signal. The difference between readings is either positive or negative. For example, as the practitioner moves the probe 100 about the predetermined area, he/she is signaled with a loud audio indication that a boundary between a tooth, or at least a portion of a tooth, and another tissue is located below the gingiva being scanned.

Optionally, a plurality of LEDs is arranged to form a graduated scale that indicates large or small differences between successive measurements. Further optionally, the graduated scale includes a plus side and a minus side, to indicate whether the measured difference is positive or negative, respectively. The added functionality that the sign of the difference is indicated, allows the practitioner to determine which side of the boundary the tooth is located on, thus allowing the practitioner to avoid the tooth with higher accuracy.

In another embodiment, the probe 100 is used to determine a specific location of a particular tissue, such as a tooth or the root of a tooth. In such cases, the difference between readings is plotted e.g., to a computer screen, to provide a visual mapping of the tissue structure below the gingiva. A practitioner scanning a predetermined region is provide with a graph, or other image, and is able to visualize the tissue structure below the predetermined region. The visual image aids the practitioner in determining a suitable location for delivering anaesthesia.

In yet another embodiment, the difference between readings is used to find regions of tissue that corresponds to regions of high density or to low density. For example, the user would like to find a region of low density corresponding to the cancellous bone adjacent the tooth requiring anaesthesia. The practitioner activates the probe 100, inserts the probe in the patients mouth, and proceeds to take a succession of readings, i.e., a first reading is measured and analyzed, followed by a second, a third, etc. The second reading is compared to the first reading to determine a plus/minus difference between the readings. Depending upon whether the difference is positive or negative, one of the readings is determined a ‘lowest’ reading. The third reading is then compared to the lowest reading, and a plus/minus difference is calculated. Again, depending upon whether the difference is plus or minus, one of these two readings is determined the lowest reading. A plurality of readings is acquired in the same manner. Each time the newest reading is calculated to be the lowest reading, an audio signal is transmitted. The intensity of the audio signal is modified according to the magnitude of the difference of the most recent reading and the lowest reading e.g., a smaller difference corresponding to a louder audio signal. When the difference between the lowest reading and the latest reading is determined to be about zero, within predetermined limits, a different characteristic audio signal is transmitted. This characteristic audio signal indicates a section of the scanned region with the lowest readings, e.g., the softest, least dense region of cancellous bone. Obviously, the more readings that are taken, the more accurate this method, within limits. Optionally, the audio signal is muted for the first five or ten readings. Alternatively, the difference in readings is used to find a ‘highest’ reading. In each if these two embodiments, the relative density difference between regions is determined.

As the reflected wave can be affected by the pressure of the contact with the tissue, it may be desirable to remove the effects using additional signal processing or additional components, as is known to those skilled in the art. However, if the pressure is constant between two successive measurements, as is likely to be the case, this will not be a significant problem since it is a difference between measurements that is measured.

In general, variations in temperature do not need to be accounted for because each successive measurement is a difference measurement.

An alternative design of the probe 100 is illustrated in FIG. 8. In this embodiment, the entire body of probe is able to fit into the patient's mouth. Since the rod 104 is tiny, there is no need for the sensing tip 102 to be tapered. The small size is advantageous in many applications. Furthermore, the small rod size lowers the probability that the rod 104 will unintentionally be placed against more than one tissue (e.g., accidentally bump the patients lip), consequently providing erroneous results.

In another embodiment, shown in FIG. 9, the probe 100 is powered with a battery 170. Optionally, the electronics module 120 is coupled to the transducer 130 via a wireless connection. The resulting portable probe is advantageously small.

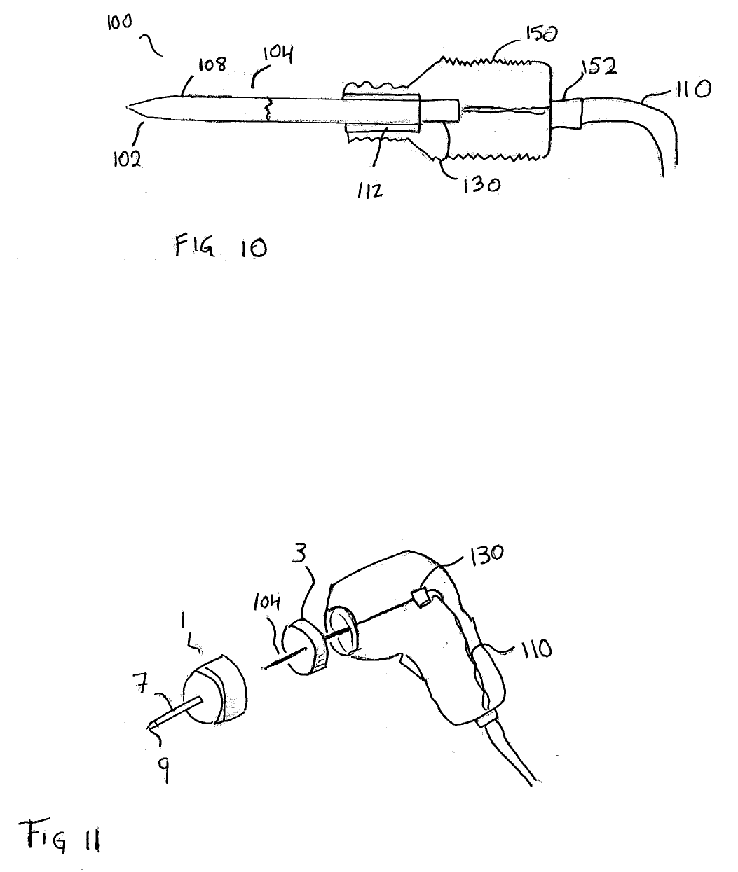

In each of the embodiments, the rod 104 is optionally flexibly supported by an annular rubber coupling 112, as shown in FIG. 10. The rubber coupling 112 lessens the rigidity of the device to provide the rod 104 with more flexibility and provides a non-conducting contact with the rod 104.

Furthermore, in each of the embodiments, the rod 104 is optionally removable. For example, the rod 104 is snapped, clamped, or screwed into the lower portion of the housing 150. The rod is thus disposable, or alternatively, sterilizable.

Alternatively, the rod is equipped with a removable tip or extension 108. The removable tip 108 becomes the sensing end 102. The removable tip is disposable and/or sterilizable. The tip can be attached in an acoustically transparent manner.

Further alternatively, the probe and/or housing is fitted with an appropriate cover. The cover provides electrical insulation to the patient and protects the probe 100 from water damage. For example, the cover is a tight fitting plastic protector or wrapping. Optionally, the cover is disposable and used to provide germ/disease protection between patients.

In another embodiment (not shown), the rod 104 is replaced with a non-rod shaped acoustic coupler. For example, a square or cylindrical covering to protect the piezoelectric crystal. In this embodiment, the piezoelectric crystal is closer to the tissue under investigation. Optionally, the acoustic coupler is disposable. For example, the acoustic coupler is in the form of a pliant cup or one-sided sleeve adapted to fit over an end of the probe 100. The acoustic coupler forms the end for coupling to the tissue in the patient's mouth.

In another embodiment (not shown), the rod 104 is substantially hollow and includes an appropriate waveguide to transmit the incident and reflected acoustic waves.

In another embodiment (not shown), the rod 104 includes a bore extending longitudinally through the rod 104 for delivery of a substance to be used to indicate the suitable location for inserting the drill/catheter. For example, in a preliminary examination, an individual having knowledge of the location of the tooth requiring anaesthesia, uses the probe 100 to determine a suitable location for inserting the drill/catheter. When a suitable location is determined, the individual actuates a fluid delivery process that causes a fluid (e.g., a blue dye) to travel through the rod 104 of the probe, where it is deposited at the suitable location. The ‘marked’ site thus serves as an indicator or markerof the suitable location for the same or another individual. Marking can be provided by other means, such as using a marking pen once the location is determined to denote the location where the absence of a tooth is determined. Alternatively, the tip of the probe can be sharp enough to be used to gently pierce the tissue so as to draw blood which can be used as a marker.

In the embodiment, shown in FIG 11, the acoustic coupler 104 is of narrow diameter, and is adapted to replace rod 27 of the drill/catheter (FIGS. 1 and 2). This embodiment has the distinct advantage that the probe 100 is in direct contact with the tissue being analyzed e.g., the cancellous bone about the tooth, thus providing more direct results. Moreover, the probe 100 is used to help direct the drill/catheter to the suitable location. Optionally, the probe 100 is triggered with a mechanism that simultaneously advances the rod 27/104 through the catheter 7 to a location where the sensing end 102 is exposed to the surrounding tissue. Further optionally, the rod 104 is equipped with a bore similar to the bore in the previous embodiment, wherein the bore serves as a fluid delivery chamber for delivering anaesthesia to the predetermined site. The probe 100 is not activated while the drill is in motion, since the rotational motion negatively affects the readings. Furthermore, the rod 104 is not secured to the adaptor 3, i.e., the rod 104 does not covey the rotational motion exhibited by the adaptor 3 to drive the perforator 1.

Many advantages of the present invention relate to the fact that the transducer is located in the housing 150 and that the incident wave(s) are transmitted through the rod 104 to the tissue. In conventional sonic probes, the transducers are located at the distal end of the probe. However, this design is not always advantageous for oral applications. Firstly, ultrasonic transducers are temperature sensitive, and thus these probes are difficult to sterilize with conventional methods. Secondly, the size of conventional ultrasonic transducers are too large to accurately determine a suitable location for delivery of medication in a patient's mouth, the tip of a conventional probe being slightly larger than a small tooth. Furthermore, the large size of these conventional probes is cumbersome and awkward for both the patient and the practitioner, when they are place within the patient's mouth.

The advantages of using a probe with a smaller, narrower acoustic coupler coupled to a transducer in the proximal end of the probe include ease of sterilization, greater durability, a more compact structure, and greater flexibility. Each of these advantages contributes to making the present invention ideal for oral applications.

Other advantages of the present invention relate to the fact that relative measurements are obtained. Since the measured readings are not compared to previously recorded and stored known data to determine characteristics of the tissue, there are significant time and money savings. In fact, the present invention provides a working model that is simpler to use and maintain. Since relative measurements are obtained, there is no need to calibrate the probe. Specifically, variations in temperature, applied pressure, similar tissues between patients, and the response of the probe due to material fatigue are not a problem.

Of course numerous other embodiments and advantages may be envisaged without departing from the spirit and scope of the invention. For example, the probe may comprise any combination of the above embodiments.

What is claimed is:

1. A method of determining a suitable target location for inserting an intaosseous device, said method comprising the steps of:
placing the probe about a first region in the individual's mouth for acquiring a first reading;
placing the probe about a second region in the individual's mouth for acquiring a second reading, the second region in close proximity to the first region;
determining a difference between the first and the second readings, the difference indicative of a difference in density between the first region and the second region; and
determining the suitable location in dependence upon the determined difference between the first and the second readings.

2. A method as defined in claim 1 further comprising the step of marking the suitable location with a marker.

3. A method as defined in claim 2 further comprising the step of inserting the intraosseous device at the target location for dispensing a fluid therethrough.

4. A method of determining a suitable location for inserting an intraosseous device comprising the steps of:
placing an electronic a probe about a plurality of locations along an upper or lower gum within a small region substantially between two teeth; and,
determining a location where the density is the least, said location being a target location where it is likely to find the absence of a tooth under the gum.

5. A method as defined in claim 4 further comprising the step of inserting the intraosseous device at the target location for dispensing a fluid therethrough.

6. A method as defined in claim 5 further comprising the step of marking the target location.
